(19) United States
(12) Patent Application Publication
(10) Pub. No.: US 2004/0220443 A1
(43) Pub. Date: Nov. 4, 2004

(54) Process for the preparation of hydrocarbons

(76) Inventors: Johannes Didericus De Graaf, Amsterdam (NL); Winnifred De Graaf, Amsterdam (NL)

Correspondence Address: Jennifer D Adamson, Shell Oil Company, Intellectual Property, PO Box 2463, Houston, TX, 77252-2463, US

(21) Appl. No.: 10/487,497

(22) PCT Filed: Aug. 9, 2002
(86) PCT No.: PCT/EP02/08950

(30) Foreign Application Priority Data
Aug. 24, 2001 (EP) 01203205.8

(51) Int. Cl. C07C 1/00

(52) U.S. Cl. 585/943

(57) ABSTRACT

A process for the preparation of liquid hydrocarbons and a clean gas stream suitable as feed and/or fuel gas from synthesis gas involving the following steps:

(i) catalytically converting the synthesis gas at elevated temperature and pressure into liquid hydrocarbons;

(ii) separating product stream obtained in step (i) into a light product stream having at least carbon dioxide, unconverted synthesis gas, light hydrocarbons, oxygenates and inerts and a heavy product stream having mainly normally liquid and normally solid hydrocarbons; and

(iii) separating at least carbon dioxide from the light product stream obtained by means of a physical absorption process using a liquid absorbent, preferably a continuous, regenerative absorption process.

The present invention relates to a process for the preparation of liquid hydrocarbons and a clean gas stream suitable as feed and/or fuel gas from synthesis gas. The invention especially relates to an efficient, integrated process for the preparation of hydrocarbons and feed and/or fuel gas, which feed and/or fuel gas is especially used for the preparation of synthesis gas and/or hydrogen, which synthesis gas and/or hydrogen, at least partially, preferably at least 50 vol %, more preferably at least 75 vol %, is preferably used in the hydrocarbon synthesis process, thus increasing the chemical efficiency, especially the carbon efficiency usually expressed as the C₃⁺ efficiency, and the energy efficiency of the overall process.

Many documents are known describing processes for the conversion of (gaseous) hydrocarbonaceous feedstocks, especially methane from natural sources, e.g. natural gas, associated gas and/or coal bed methane, into liquid and optionally solid products, especially methanol and liquid hydrocarbons, particularly paraffinic hydrocarbons. In these documents reference is often made to remote locations and/or off-shore locations, where no direct use of the gas is possible. Transportation of the gas, e.g. through a pipeline or in the form of liquefied natural gas, requires extremely high capital expenditure or is simply not practical. This holds even more in the case of relatively small gas production rates and/or gas fields. Reinjection of associated gas may add to the costs of oil production, and may result in undesired effects on the crude oil production. Burning of associated gas has become an undesired option in view of depletion of hydrocarbon sources and air pollution.

A process often used for the conversion of carbonaceous feedstocks into liquid and/or solid hydrocarbons is the well-known Fischer-Tropsch process.

In WO 94/21512 a process for the production of methanol has been described from an off-shore natural gas field using a floating platform. However, no integrated, efficient, low-cost process scheme has been described.

In WO 97/12118 a method and system for the treatment of a well stream from an off-shore oil and gas field has been described. Natural gas is converted into syngas using pure oxygen in an autothermal reformer, a combination of partial oxidation and adiabatic steam reforming. The syngas (comprising a considerable amount of carbon dioxide) is converted into liquid hydrocarbons and wax. No fully integrated process scheme for a highly efficient, low capital process is described in this document.

In WO 91/15446 a process is described to convert natural gas, particularly remote location natural gas (including associated gas), in the form of normally liquid hydrocarbons suitable for fuel use via methanol/dimethyl ether. However, no integrated, efficient, low-cost process scheme has been described.

In U.S. Pat. No. 4,833,170 a process is described for the production of heavier hydrocarbons from one or more gaseous hydrocarbons. The gaseous hydrocarbons are converted into syngas by autothermal reforming with air in the presence of recycled carbon dioxide and steam. However, no (energy) integrated, efficient, low-cost process scheme has been described.

In CA 1,288,781 a process for the production of liquid hydrocarbons has been described comprising the steps of catalytically reforming the hydrocarbonaceous feed, heating the reforming zone by means of a carbon dioxide-containing heating gas comprising a product which has been obtained by partial oxidation of reformer product, separating carbon dioxide from the heating gas, catalytically converting the reformer product after separating off carbon dioxide into liquid hydrocarbons and combining the carbon dioxide obtained above with the hydrocarbonaceous feed used in the catalytic reforming process.

An object of the present invention is to provide an improved scheme for the production of especially (easily manageable) normally liquid hydrocarbons (S.T.P.) and normally solid hydrocarbons (S.T.P.) from a hydrocarbonaceous feedstock, especially light hydrocarbons as natural or associated gas, together with a light product in the form of a clean gas stream suitable as feed and/or fuel gas, which feed and/or fuel gas may be used especially for the preparation of synthesis gas and/or hydrogen.

It is observed that the Fischer-Tropsch hydrocarbon synthesis process always results in the desired liquid and optionally solid hydrocarbons, together with a light product stream comprising saturated C₁-C₄ hydrocarbons, unsaturated C₂-C₄ hydrocarbons, unconverted synthesis gas, carbon dioxide, inerts (mainly nitrogen and argon), a minor amount of C₅⁺ hydrocarbons (as the separation between C₄⁻ and C₅⁺ usually is not perfect) and some oxygenates (mainly C₂-C₄ alcohols, dimethyl ether and some lower (C₁ to C₄) aldehydes/ketones).

Carbon dioxide is an undesired product in the product streams obtained in the Fischer-Tropsch reaction. It is especially formed when an iron based catalyst is used, but also the use of cobalt based catalyst may result in the formation of small amounts of carbon dioxide. However, the use of cobalt in combination with certain promoters (to enhance specific product properties) may result in the formation of larger amounts of carbon dioxide. Also the use of recycle streams may result is synthesis gas streams comprising substantial amount of carbon dioxide (e.g. between 1 and 30 vol %, often between 3 and 25 vol %). Another source of carbon dioxide is the carbon dioxide present in the synthesis gas stream used for the FT synthesis. Usually the synthesis gas contains a few percent of carbon dioxide. The present invention in particular concerns the removal of carbon dioxide from gas streams obtained after the heavy hydrocarbon synthesis reaction (Fischer-Tropsch reaction), optionally in combination with similar processes to remove carbon dioxide form the main synthesis gas stream for the Fischer-Tropsch reaction. In particular a physical absorption process is to be used, rather than a chemical process. The physical process also removes larger hydrocarbon molecules, including unsaturates. This may improve the process efficiency. Further, physical processes also remove part of the inerts (nitrogen, argon) which may improve the FT performance when removed from a recycle stream.

In the past it has often been suggested to use the untreated light product stream as a feed and/or fuel gas to generate synthesis gas and/or hydrogen and energy.

There are, however a number of disadvantages in using this untreated light stream as fuel. Firstly, due the usually high amounts of carbon dioxide, the caloric value is relatively low. The use of such low caloric value fuel is not efficient. Secondly, the presence of unsaturated compounds may result in the (quick) fouling of the burners due to the formation of coke. This makes regular cleaning necessary, and lowers the efficiency of the burner Further, it has also been suggested to use this light product stream as feed for a steam-methane reforming process. However, due to the presence of carbon monoxide, unsaturated compounds and some C₅⁺ compounds, this is hardly possible as each of these compounds results in the formation of coke deposits on the catalyst. In addition, the presence of high amounts of carbon dioxide results in a relatively low hydrogen/carbon monoxide ratio. Also the use of this light product stream as feed for a (catalytic) partial oxidation reaction (or any combination of steam methane reforming/(catalytic) partial oxidation) in order to produce synthesis gas is not a very attractive solution in view of the high carbon dioxide amount, resulting in a relatively low hydrogen/carbon monoxide ratio.

It has now been found that treatment of the light product stream by means of a continuous, regenerative, physical absorption process using a liquid absorbent results in a treated gas stream from which all or almost all of the carbon dioxide and substantially all of the unsaturated compounds, oxygenates and the heavier hydrocarbons (especially the C₄⁺ fraction) have been removed. This means that a clean fuel gas is obtained having a considerable increased caloric value, while also components which may cause problems as coke formation have been removed. Thus, the applicability of the light products stream has been considerably improved while also valuable products are recovered.

The present invention therefore relates to a process as described in claim 1.

The hydrocarbon synthesis as mentioned in step (i) of the present invention may be any suitable hydrocarbon synthesis step known to the man skilled in the art, but is preferably a Fischer-Tropsch reaction. The synthesis gas to be used for the hydrocarbon synthesis reaction, especially the Fischer-Tropsch reaction, is made from a hydrocarbonaceous feed, especially by partial oxidation, catalytic partial oxidation and/or steam/methane reforming. In a suitable embodiment an autothermal reformer is used or a process in which the hydrocarbonaceous feed is introduced into a reforming zone, followed by partial oxidation of the product thus obtained, which partial oxidation product is used for heating the reforming zone. The hydrocarbonaceous feed is suitably methane, natural gas, associated gas or a mixture of C₁₋₄ hydrocarbons, especially natural gas.

To adjust the H₂/CO ratio in the syngas, carbon dioxide and/or steam may be introduced into the partial oxidation process and/or reforming process. The H₂/CO ratio of the syngas is suitably between 1.3 and 2.3, preferably between 1.6 and 2.1. If desired, (small) additional amounts of hydrogen may be made by steam methane reforming, preferably in combination with the water-gas shift reaction. The additional hydrogen may also be used in other processes, e.g. hydrocracking.

The synthesis gas obtained in the way as described above, usually having a temperature between 900 and 1400° C., is cooled to a temperature between 100 and 500° C., suitably between 150 and 450° C., preferably between 300 and 400° C., preferably under the simultaneous generation of power, e.g. in the form of steam. Further cooling to temperatures between 40 and 130° C., preferably between 50 and 100° C., is done in a conventional heat exchanger, especially a tubular heat exchanger. In another embodiment at least part of the cooling is obtained by quenching with water.

The purified gaseous mixture, comprising predominantly hydrogen and carbon monoxide, is contacted with a suitable catalyst in the catalytic conversion stage, in which the normally liquid hydrocarbons are formed.

The catalysts used for the catalytic conversion of the mixture comprising hydrogen and carbon monoxide into hydrocarbons are known in the art and are usually referred to as Fischer-Tropsch catalysts. Catalysts for use in this process frequently comprise, as the catalytically active component, a metal from Group VIII of the Periodic Table of Elements. Particular catalytically active metals include ruthenium, iron, cobalt and nickel. Cobalt is a preferred catalytically active metal.

The catalytically active metal is preferably supported on a porous carrier. The porous carrier may be selected from any of the suitable refractory metal oxides or silicates or combinations thereof known in the art. Particular examples of preferred porous carriers include silica, alumina, titania, zirconia, ceria, gallia and mixtures thereof, especially silica, alumina and titania.

The amount of catalytically active metal on the carrier is preferably in the range of from 3 to 300 pbw per 100 pbw of carrier material, more preferably from 10 to 80 pbw, especially from 20 to 60 pbw.

If desired, the catalyst may also comprise one or more metals or metal oxides as promoters. Suitable metal oxide promoters may be selected from Groups IIA, IIIB, IVB, VB and VIB of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are very suitable promoters. Particularly preferred metal oxide promoters for the catalyst used to prepare the waxes for use in the present invention are manganese and zirconium oxide. Suitable metal promoters may be selected from Groups VIIB or VIII of the Periodic Table. Rhenium and Group VIII noble metals are particularly suitable, with platinum and palladium being especially preferred. The amount of promoter present in the catalyst is suitably in the range of from 0.01 to 100 pbw, preferably 0.1 to 40, more preferably 1 to 20 pbw, per 100 pbw of carrier. The most preferred promoters are selected from vanadium, manganese, rhenium, zirconium and platinum.

The catalytically active metal and the promoter, if present, may be deposited on the carrier material by any suitable treatment, such as impregnation, kneading and extrusion. After deposition of the metal and, if appropriate, the promoter on the carrier material, the loaded carrier is typically subjected to calcination. The effect of the calcination treatment is to remove crystal water, to decompose volatile decomposition products and to convert organic and inorganic compounds to their respective oxides. After calcination, the resulting catalyst may be activated by contacting the catalyst with hydrogen or a hydrogen-containing gas, typically at temperatures of about 200 to 350° C. Other processes for the preparation of Fischer-Tropsch catalysts comprise kneading/mulling, often followed by extrusion, drying/calcination and activation.

The catalytic conversion process may be performed under conventional synthesis conditions known in the art. Typically, the catalytic conversion may be effected at a temperature in the range of from 150 to 300° C., preferably from 180 to 260° C. Typical total pressures for the catalytic conversion process are in the range of from 1 to 200 bar absolute, more preferably from 10 to 70 bar absolute. In the catalytic conversion process especially more than 75 wt % of C₅⁺, preferably more than 85 wt % C₅⁺ hydrocarbons are formed. Depending on the catalyst and the conversion conditions, the amount of heavy wax (C₂₀⁺) may be up to 60 wt %, sometimes up to 70 wt %, and sometimes even up till 85 wt %. Preferably a cobalt catalyst is used, a low H₂/CO ratio is used and a low temperature is used (190-230° C.). To avoid any coke formation, it is preferred to use an H₂/CO ratio of at least 0.3. It is especially preferred to carry out the Fischer-Tropsch reaction under such conditions that the SF-alpha value, for the obtained products having at least 20 carbon atoms, is at least 0.925, preferably at least 0.935, more preferably at least 0.945, even more preferably at least 0.955.

Preferably, a Fischer-Tropsch catalyst is used, which yields substantial quantities of paraffins, more preferably substantially unbranched paraffins. A most suitable catalyst for this purpose is a cobalt-containing Fischer-Tropsch catalyst. Such catalysts are described in the literature, see e.g. AU 698392 and WO 99/34917.

The Fischer-Tropsch process may be a slurry FT process or a fixed bed FT process, especially a multitubular fixed bed.

The physical adsorption process to be used in the process of the present invention is well known to the man skilled in the art. Reference can be made to e.g. Perry, Chemical Engineerings' Handbook, Chapter 14, Gas Absorption. The absorption process to be used in the present process is a physical process. Suitable solvents are well known to the man skilled in the art and are described in the literature. In the present process the liquid absorbent in the physical absorption process is suitably methanol, ethanol, acetone, dimethyl ether, methyl i-propyl ether, polyethylene glycol or xylene, preferably methanol. The physical absorption process is suitably carried out at relatively low temperatures, preferably between −60° C. and 50° C., preferably between −30 and −10° C.

The physical absorption process is carried out by contacting the light products stream in a counter-current upward flow with the liquid absorbent. The absorption process is preferably carried out in a continuous mode, in which the liquid absorbent is regenerated. This regeneration process is well known to the man skilled in the art. The loaded liquid absorbent is suitably regenerated by pressure release (e.g. a flashing operation) and/or temperature increase (e.g. a distillation process). The regeneration is suitably carried out in two or more steps, preferably 3-10 steps, especially a combination of one or more flashing steps and a distillation step.

The light hydrocarbons in the light product stream especially comprise C₁ to C₆ hydrocarbons, preferably C₁ to C₅ hydrocarbons, more preferably C₁ to C₄ hydrocarbons, and the heavy product stream comprises suitably all the C₆⁺ hydrocarbons, preferably also the C₅⁺ hydrocarbons. It is observed that the light products stream preferably comprises the normally gaseous hydrocarbons (i.e. the C₁ to C₄ hydrocarbons), and the heavy product stream comprises mainly the normally liquid and (optionally) normally solid hydrocarbons (i.e. the C₅⁺ hydrocarbons). Depending on the conditions in the actual separation process, however, the light fraction will comprise some of the heavy products and the heavy product fraction will comprise some of the light products.

When carrying out the physical absorption process of the present invention, not only carbon dioxide will be removed, but also a part, preferably a substantial part, e.g. at least 50 wt %, preferably at least 75 wt %, of the hydrocarbons present in the light product stream will be removed. The absorbed hydrocarbons are mainly C₃ to C₆ hydrocarbons, preferably C₄ to C₅, although also some C₇⁺ hydrocarbons may be present. These hydrocarbons may be isolated from the absorbent liquid, and especially the C₅⁺ hydrocarbons may be added to the hydrocarbon products stream. Hydrogen and carbon monoxide are hardly absorbed in the physical absorption process to be used in the present invention. Part of the ethane, preferably less than 50 vol %, more preferably less than 75 vol %, is removed in the absorption process.

At least part of the treated light product stream may be used for the preparation of synthesis gas. This synthesis gas is preferably used in the preparation of hydrocarbons according to step (i) of the present process as this enhances the overall carbon yield of the process. In that case the treated light product stream may be converted in a separate synthesis gas plant (e.g. (catalytical) partial oxidation, steam methane reforming, autothermal reforming etc.) or may be mixed with the main hydrocarbonaceous feed for the synthesis gas manufacture. The second option is the preferred method as it will be the more efficient way. Carbon dioxide may also be removed from the synthesis gas stream obtained in that way, from the dedicated syngas manufacturing unit as well as from the main synthesis gas stream obtained after oxidation and/or reforming the combined feed stream. It is observed that it is an additional advantage that the regeneration of the physical solvent used in the above process may be combined with the regeneration of the physical process used in step (iii) of the process according to the invention. Please note that when the synthesis gas stream is treated with a physical absorption process also compounds as HCN, COS and H₂S are removed beside the carbon dioxide. This obviates a sulphur removal process of the gaseous hydrocarbonaceous fees stream. Especially when different types of organic sulphur compounds are present, this is an additional advantage (simplicity, carbon efficiency).

Part of the treated light product stream may also-be used in the production of synthesis gas or hydrogen in a steam hydrocarbon reforming reaction, preferably as feed stream as this enhances the overall carbon yield of the process The gas stream obtained contains a relatively high amount of hydrogen, and may, optionally after CO removal/conversion, be used for several purposes, e.g. product work-up (catalytical hydrogenation, isomerization, hydrocracking, hydrofinishing), adjustment of the H₂/CO ratio in the Fischer-Tropsch process, desulphurisation of feedstreams etc. It is observed that it is an additional advantage that the regeneration of the physical solvent used in the above process may be combined with the regeneration of the physical process used in step (iii) of the process according to the invention. Please note that in the case that CO₂ is removed from one or more Fischer-Tropsch recycle streams, also here regeneration of the loaded solvent may be combined with other regeneration operations, especially the regeneration of the physical process used in step (iii) of the process according to the invention.

In another embodiment the invention further relates to a process for the preparation of hydrocarbons from synthesis gas comprising the following steps:

(i) partial oxidation optionally in combination with steam methane reforming of a hydrocarbonaceous feed resulting in a synthesis gas having a relatively low hydrogen/carbon monoxide ratio;

(ii) steam hydrocarbon reforming of another hydrocarbonaceous feed resulting in a synthesis gas having a relatively high hydrogen/carbon monoxide ratio:

(iii) using the synthesis gas obtained in steps (i) and (ii) in a catalytic conversion process in which the synthesis gas is converted at elevated temperature and pressure into liquid hydrocarbons, in which process carbon dioxide is removed from the synthesis gas obtained in step (ii) by means of a (continuous, regenerative,) physical absorption process using a liquid absorbent. Such a process may be combined with the process as described in claim 1 of the present invention, especially when the regeneration units are combined.

1. A process for the preparation of liquid hydrocarbons and a clean gas stream suitable as feed and/or fuel gas from synthesis gas comprising the following steps:
(i) catalytically converting the synthesis gas at elevated temperature and pressure into a product stream comprising liquid hydrocarbons;
(ii) separating the product stream obtained in step (i) into a light product stream comprising at least carbon dioxide, unconverted synthesis gas, light hydrocarbons, oxygenates and inerts and a heavy product stream comprising mainly normally liquid and normally solid hydrocarbons;
(iii) separating at least carbon dioxide from the light product stream by means of a physical absorption process using a liquid absorbent to produce a treated light product stream; and
wherein at least part of the treated light product stream is used for the production of synthesis gas.

2. The process of according to claim 1, in which the liquid absorbent in the physical absorption process is selected from the group consisting of methanol, ethanol, acetone, dimethyl ether, methyl i-propyl ether, polyethylene glycol and xylene.

3. The process of claim 1, in which the physical absorption process is carried out by contacting the light products stream in a counter-current upward flow with the liquid absorbent.

4. The process of claim 1, in which the light hydrocarbons in the light product stream comprise C₁ to C₆ hydrocarbons, and the heavy product stream comprises C₆⁺ hydrocarbons.

5. The process of claim 1, in which the absorbed hydrocarbons are mainly C₃ to C₆ hydrocarbons.

6. The process of claim 1, in which the synthesis gas is used for the preparation of hydrocarbons according to step (i) of the present process.

7. The process of claim 1, in which at least part of the treated light product stream is used in the production of synthesis gas or hydrogen in a steam hydrocarbon reforming reaction.

8. The process of claim 6, in which carbon dioxide is removed from the synthesis gas by means of a continuous, regenerative physical absorption process using a liquid absorbent.

9. The process of claim 1, in which the catalyst used in step (i) is a cobalt based catalyst.

10. The process of claim 1, in which in step (iii) at least 50 vol % of the carbon dioxide is removed.
